(19) United States
(12) Patent Application Publication
(10) Pub. No.: US 2026/0314939 A1
(43) Pub. Date: Oct. 15, 2026

(54) PHYSICAL CONDITION DIAGNOSIS DEVICE, WEARABLE DEVICE, PHYSICAL CONDITION DIAGNOSIS SYSTEM, MODEL DETERMINATION DEVICE, AND PHYSICAL CONDITION DIAGNOSIS METHOD

(71) Applicant: MITSUBISHI ELECTRIC CORPORATION, Tokyo (JP)

(72) Inventors: Hikaru MIYAUCHI, Tokyo (JP); Izaya NISHIMUTA, Tokyo (JP); Kenta KINEMUCHI, Tokyo (JP); Noritaka OKUDA, Tokyo (JP); Yuka TSUDA, Tokyo (JP); Kazuya TAKAKI, Tokyo (JP)

(73) Assignee: MITSUBISHI ELECTRIC CORPORATION, Tokyo (JP)

(21) Appl. No.: 19/484,387

(22) PCT Filed: Jun. 14, 2023
(86) PCT No.: PCT/JP2023/022164

§ 371 Date: Nov. 14, 2025

(51) Int. Cl. A61B 5/01; A61B 5/00; A61B 5/11

(52) U.S. Cl. CPC A61B 5/01; A61B 5/0008; A61B 5/1118; A61B 5/6803; A61B 5/7246; A61B 5/7264

(57) ABSTRACT

A physical condition diagnosis device includes an information acquirer, an activity-related body temperature estimator, and a physical condition diagnoser. The information acquirer acquires vital sign information and variation factor information. The vital sign information includes temperature information of at least one part of a subject organism to undergo physical condition diagnosis. The variation factor information includes activity information indicating an activity of the subject organism causing temperature variations in the at least one part. The activity-related body temperature estimator determines, based on the vital sign information and the variation factor information, activity-related body temperature data indicating the temperature variations in the at least one part caused by the activity. The physical condition diagnoser determines a physical condition index indicating a physical condition of the subject organism based on the activity-related body temperature data.

### TECHNICAL FIELD

The present disclosure relates to a physical condition diagnosis device, a wearable device, a physical condition diagnosis system, a model determination device, a physical condition diagnosis method, and a program.

### BACKGROUND ART

For physical condition management, techniques have been developed to, for example, predict the physical conditions of subject organisms based on vital sign information. As an example of such a technique, Patent Literature 1 describes a biorhythm prediction device that predicts an ultradian rhythm waveform of vital sign information, such as the body temperature or the heart rate index of a user. The biorhythm prediction device described in Patent Literature 1 reduces the degree by which causal factors affect a biosignal with an ultradian rhythm cycle, and predicts the ultradian rhythm waveform during a predetermined action of the user to reflect the predetermined action.

As another example, Patent Literature 2 describes a physical condition monitoring device that evaluates an abnormality state of a body based on vital sign information, such as blood pressure, pulse, and body temperature. The physical condition monitoring device described in Patent Literature 2 corrects a reference value based on the mental or physical activity state of the subject, and determines whether the body has an abnormality based on comparison between the measurement value of the vital sign information and the corrected reference value.

### CITATION LIST

### Patent Literature

Patent Literature 1: Unexamined Japanese Patent Application Publication No. 2021-49041
Patent Literature 2: Unexamined Japanese Patent Application Publication No. H4-71532

### SUMMARY OF INVENTION

### Technical Problem

Although the biorhythm prediction device described in Patent Literature 1 can predict the ultradian rhythm waveform during a predetermined action of the user to reflect the predetermined action, the device cannot diagnose the physical condition of the subject.

The physical condition monitoring device described in Patent Literature 2 determines whether the body has an abnormality based on comparison between a measurement value of vital sign information, such as body temperature, and the corrected reference value. Thus, the physical condition monitoring device described in Patent Literature 2 can diagnose an abnormality based on body temperature variations when the abnormality in the body causes major variations in the body temperature as the dominant factor in the variations. In other words, the physical condition monitoring device described in Patent Literature 2 cannot easily detect an abnormality in the body when the abnormality is not the dominant factor in the body temperature variations. The physical condition of the subject organism thus cannot be diagnosed accurately.

Under such circumstances, an objective of the present disclosure is to provide a physical condition diagnosis device, a wearable device, a physical condition diagnosis system, a model determination device, a physical condition diagnosis method, and a program that can diagnose the physical condition of a subject organism accurately.

### Solution to Problem

To achieve the above objective, a physical condition diagnosis device according to an aspect of the present disclosure includes an information acquirer, an activity-related body temperature estimator, and a physical condition diagnoser. The information acquirer acquires vital sign information and variation factor information. The vital sign information includes temperature information of at least one part of a subject organism to undergo physical condition diagnosis. The variation factor information includes activity information indicating an activity of the subject organism causing temperature variations in the at least one part. The activity-related body temperature estimator determines, based on the vital sign information and the variation factor information, activity-related body temperature data indicating the temperature variations in the at least one part caused by the activity. The physical condition diagnoser determines a physical condition index indicating a physical condition of the subject organism based on the activity-related body temperature data.

### Advantageous Effects of Invention

The physical condition diagnosis device according to the above aspect of the present disclosure determines the physical condition index of the subject organism based on the activity-related body temperature data indicating temperature variations in a part of the subject organism caused by an activity of the subject organism. The physical condition of the subject organism can thus be diagnosed accurately.

### BRIEF DESCRIPTION OF DRAWINGS

FIG. 1 is a block diagram of a physical condition diagnosis system according to Embodiment 1;

FIG. 2 is a diagram of a physical condition diagnosis device according to Embodiment 1, illustrating the hardware configuration;

FIG. 3 is a flowchart of an example operation of a physical condition diagnosis process performed by the physical condition diagnosis device according to Embodiment 1;

FIG. 4 is a timing chart illustrating examples of temperature information, activity information, and activity-related body temperature data in Embodiment 1;

FIG. 5 is a diagram illustrating an example display of the diagnosis results determined by the physical condition diagnosis device according to Embodiment 1;

FIG. 6 is a block diagram of a physical condition diagnosis system according to Embodiment 2;

FIG. 7 is a timing chart illustrating examples of the temperature information and the activity information in Embodiment 2;

FIG. 8 is a flowchart of an example operation of a process of calculating the activity-related body temperature data performed by a physical condition diagnosis device according to Embodiment 2;

FIG. 9 is a timing chart illustrating an example of the activity-related body temperature data in Embodiment 2;

FIG. 10 is a diagram illustrating an example of a heat transfer path in the body of a subject organism in Embodiment 2;

FIG. 11 is a diagram illustrating an example display of the diagnosis results determined by the physical condition diagnosis device according to Embodiment 2;

FIG. 12 is a block diagram of a physical condition diagnosis system according to Embodiment 3;

FIG. 13 is a diagram illustrating an example display of the diagnosis results determined by a physical condition diagnosis device according to Embodiment 3;

FIG. 14 is a block diagram of a physical condition diagnosis system according to Modification 1 of the embodiments;

FIG. 15 is a block diagram of a physical condition diagnosis system according to Modification 2 of the embodiments;

FIG. 16 is a block diagram of a physical condition diagnosis system according to Modification 3 of the embodiments;

FIG. 17 is a block diagram of a physical condition diagnosis system according to Modification 4 of the embodiments; and

FIG. 18 is a block diagram of a physical condition diagnosis device according to a modification of the embodiments, illustrating the hardware configuration.

### DESCRIPTION OF EMBODIMENTS

A physical condition diagnosis device, a wearable device, a physical condition diagnosis system, a model determination device, a physical condition diagnosis method, and a program according to one or more embodiments of the present disclosure are described in detail below with reference to the accompanying drawings. In the drawings, the elements identical or corresponding to each other are assigned the same reference sign.

### Embodiment 1

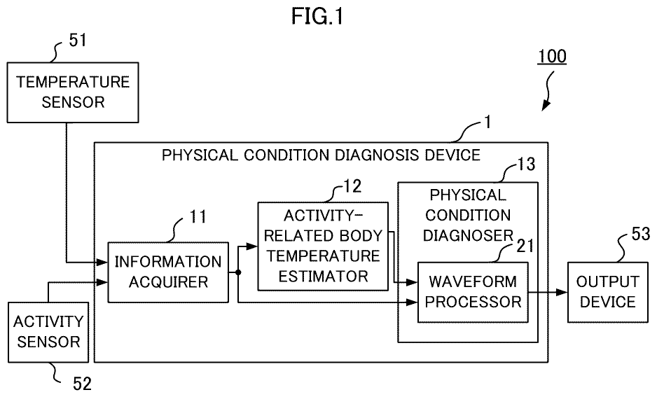

A physical condition diagnosis device that performs physical condition diagnosis for an organism such as a human or an animal, and a physical condition diagnosis system including the physical condition diagnosis device according to Embodiment 1 are described. The organism to undergo physical condition diagnosis is referred to as a subject organism. A physical condition diagnosis system 100 illustrated in FIG. 1 includes a temperature sensor 51, an activity sensor 52, a physical condition diagnosis device 1, and an output device 53. The temperature sensor 51 measures the temperature of at least one part of the subject organism. The activity sensor 52 generates an activity index being a numerical value indicating the activity of the subject organism causing temperature variations in the part. The physical condition diagnosis device 1 determines a physical condition index indicating the physical condition of the subject organism based on the measurement results acquired by the temperature sensor 51 and the activity index generated by the activity sensor 52. The output device 53 outputs the diagnosis results including the physical condition index determined by the physical condition diagnosis device 1.

The temperature sensor 51 is installed inside or outside the body of the subject organism to measure the temperature of at least one part of the subject organism. The temperature of a part of the subject organism may be the temperature of any part of the body that can be measured or estimated, and includes a body surface temperature as well as a body inside temperature such as an organ temperature or a core temperature. The temperature sensor 51 includes, for example, a temperature sensor attached to an armpit, a calf, or an upper arm of the subject organism to undergo physical condition diagnosis, a temperature sensor installed in the stomach of a livestock animal to undergo physical condition diagnosis, and an infrared camera or a thermography installed apart from the subject organism. The temperature sensor attached to the subject organism may be, for example, a thermistor or a resistance temperature detector. In Embodiment 1, the temperature sensor 51 is attached to an armpit of the subject organism to undergo physical condition diagnosis.

The activity sensor 52 generates the activity index being a numerical value indicating an activity of the subject organism, such as eating, exercise, sleep, and excretion. The activity sensor 52 includes, for example, a wearable terminal that measures the heart rate and the moving speed of the subject organism, a camera that captures images of the subject organism, and a position sensor that acquires the position of the subject organism. In Embodiment 1, the activity sensor 52 is a position sensor that detects the subject organism to undergo physical condition diagnosis at an eating location. When the position sensor detects the subject organism at the location, the subject organism can be determined to be eating.

The physical condition diagnosis device 1 includes an information acquirer 11, an activity-related body temperature estimator 12, and a physical condition diagnoser 13. The information acquirer 11 acquires vital sign information and variation factor information. The vital sign information includes temperature information of at least one part of the subject organism. The variation factor information includes activity information indicating the activity of the subject organism causing temperature variations in the part. The activity-related body temperature estimator 12 determines activity-related body temperature data indicating the temperature variations in the part of the subject organism caused by the activity of the subject organism. The physical condition diagnoser 13 determines the physical condition index indicating the physical condition of the subject organism based on the activity-related body temperature data.

The information acquirer 11 acquires the measurement results from the temperature sensor 51 as the temperature information, and acquires the activity index generated by the activity sensor 52 as the activity information. The information acquirer 11 transmits the vital sign information including the temperature information to the activity-related body temperature estimator 12, and transmits the variation factor information including the activity information to the activity-related body temperature estimator 12 and the physical condition diagnoser 13.

The activity-related body temperature estimator 12 determines, based on the vital sign information and the variation factor information, the activity-related body temperature data indicating the temperature variations in a part of the subject organism caused by the activity of the subject organism. In an example, the activity-related body temperature estimator 12 determines the activity-related body temperature data indicating the temperature variations in the stomach during a meal.

The physical condition diagnoser 13 includes a waveform processor 21 that diagnoses the physical condition of the subject organism by performing waveform processing on the activity-related body temperature data.

The waveform processor 21 determines the physical condition index based on at least one of the phase characteristic, the amplitude, the frequency spectrum, or the waveform of the waveform data of the activity-related body temperature data.

The output device 53 receives the physical condition index from the physical condition diagnoser 13. The output device 53 outputs at least one of visual or audio information indicating the received physical condition index or a control signal corresponding to the physical condition index for an external device.

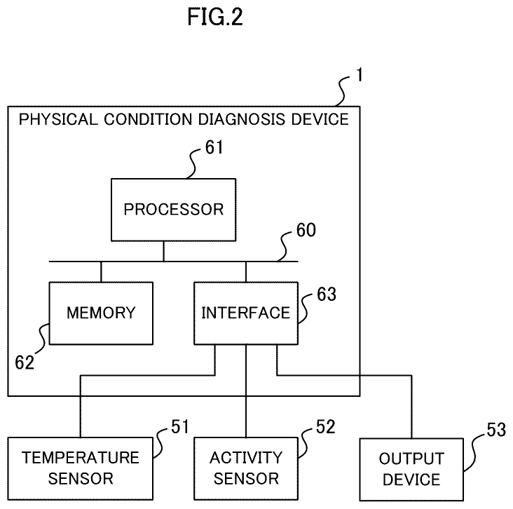

FIG. 2 illustrates hardware configuration of the physical condition diagnosis device 1 with the above structure. The physical condition diagnosis device 1 includes a processor 61, a memory 62, and an interface 63. The processor 61, the memory 62, and the interface 63 are connected to one another with a bus 60. The function of each component of the physical condition diagnosis device 1 is implemented by software, firmware, or a combination of software and firmware. The software and the firmware are written as programs and are stored in the memory 62. The processor 61 reads and executes the programs stored in the memory 62 to implement the function of each component described above. In other words, the memory 62 stores the programs for performing the processing of each component of the physical condition diagnosis device 1.

The memory 62 includes, for example, a nonvolatile or volatile semiconductor memory such as a random-access memory (RAM), a read-only memory (ROM), a flash memory, an erasable programmable ROM (EPROM), or an electrically erasable programmable ROM (EEPROM), a magnetic disk, a flexible disk, an optical disc, a compact disc (CD), a minidisc, or a digital versatile disc (DVD).

The physical condition diagnosis device 1 is connected to the temperature sensor 51, the activity sensor 52, and the output device 53 through the interface 63. The interface 63 includes an interface module compliant with one or more standards as appropriate for a connection destination.

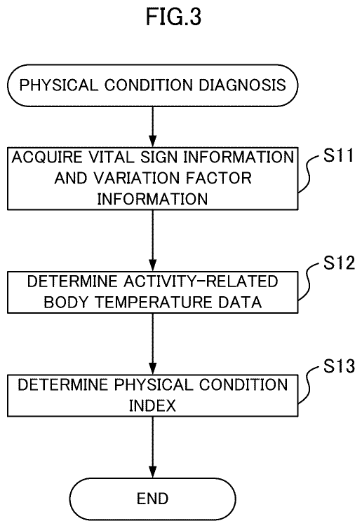

After being activated, the physical condition diagnosis device 1 with the above structure repeats a process of physical condition diagnosis illustrated in FIG. 3 at defined intervals. The information acquirer 11 included in the physical condition diagnosis device 1 acquires the vital sign information and the variation factor information (step S11). The vital sign information includes the temperature information indicating the measurement results acquired by the temperature sensor 51. The variation factor information includes the activity information including the activity index generated by the activity sensor 52.

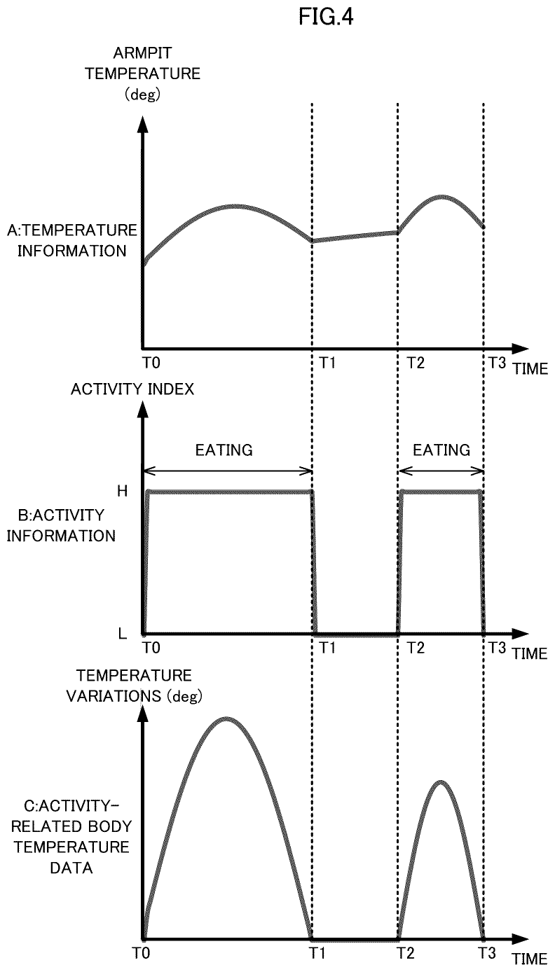

Graph A in FIG. 4 illustrates an example of the temperature information included in the vital sign information acquired by the information acquirer 11. In graph A in FIG. 4, the horizontal axis indicates time, and the vertical axis indicates armpit temperature (in degrees). Graph B in FIG. 4 illustrates an example of the activity information included in the variation factor information acquired by the information acquirer 11. In graph B in FIG. 4, the horizontal axis indicates time, and the vertical axis indicates the activity index generated by the activity sensor 52. The activity index indicates a high (H) level or a low (L) level. The activity index generated by the activity sensor 52 is an H level when the subject organism to undergo physical condition diagnosis is at the eating location, and is an L level when the subject organism is not at the eating location. When the activity index acquired by the information acquirer 11 from the activity sensor 52 is an H level, the subject organism can be determined to be eating, as illustrated in graph B in FIG. 4. When the activity index acquired by the information acquirer 11 from the activity sensor 52 is an L level, the subject organism can be determined not to be eating.

The information acquirer 11 transmits the vital sign information and the variation factor information to the activity-related body temperature estimator 12, and transmits the variation factor information to the physical condition diagnoser 13.

As illustrated in FIG. 3, the activity-related body temperature estimator 12 determines, based on the vital sign information and the variation factor information, the activity-related body temperature data indicating the temperature variations in a part of the subject organism caused by the activity of the subject organism (step S12). The activity of the subject organism includes a mental activity and a physical activity of the subject organism. The association between the activity of the subject organism and the part of the subject organism that undergoes temperature variations caused by the activity is acquired based on biological knowledge. For example, an activity of the subject organism is associated with, based on biological knowledge, an organ that undergoes heat production or heat dissipation caused by the activity.

In Embodiment 1, the activity-related body temperature estimator 12 determines the temperature variations in the stomach of the subject organism in a period in which the subject organism is eating as indicated by the activity information included in the variation factor information. When the activity index illustrated in FIG. 4 is an H level indicating that the subject organism is eating, or specifically, in the period from time T0 to time T1 and the period from time T2 to time T3, the activity-related body temperature estimator 12 extracts a variation component from the temperature information. While the subject organism is eating, the stomach temperature increases due to the digestive activity. Thus, the variation component acquired from the temperature information while the subject organism is eating corresponds to the temperature variations in the stomach.

As described above, the activity-related body temperature estimator 12 extracts the variation component from the temperature information while the activity index is an H level. The activity-related body temperature estimator 12 thus determines the activity-related body temperature data indicating the temperature variations in the stomach of the subject organism during a meal, as illustrated in graph C in FIG. 4. In graph C in FIG. 4, the horizontal axis indicates time, and the vertical axis indicates the temperature variations in the stomach (in degrees). The activity-related body temperature estimator 12 transmits the determined activity-related body temperature data to the physical condition diagnoser 13.

As illustrated in FIG. 3, the physical condition diagnoser 13 included in the physical condition diagnosis device 1 determines the physical condition index of the subject organism based on the activity-related body temperature data acquired from the activity-related body temperature estimator 12 and the activity information acquired from the information acquirer 11 (step S13). The physical condition diagnoser 13 diagnoses the physical condition of the subject organism by analyzing a single organ or multiple organs contributing to a specific function. More specifically, the physical condition diagnoser 13 determines the physical condition index indicating at least one of whether the subject organism is in a healthy condition, whether the subject organism has a disease, or the health level of the subject organism indicated by a numerical value. In Embodiment 1, the physical condition diagnoser 13 determines the stomach health level and the stomach metabolism as the physical condition index.

As illustrated in FIG. 1, the waveform processor 21 included in the physical condition diagnoser 13 acquires the activity information from the information acquirer 11 and the activity-related body temperature data from the activity-related body temperature estimator 12. The waveform processor 21 performs the fast Fourier transform (FFT) on the activity-related body temperature data to generate frequency domain data, and determines whether the frequency corresponding to the peak value of the frequency domain data is within a target range. The target range may be defined based on biological knowledge. When the frequency corresponding to the peak value is within the target range, the digestive function and organs associated with digestion can be determined to be healthy. When the frequency corresponding to the peak value is out of the target range, the digestive function or the organs associated with digestion can be determined to be likely to have a disease.

In Embodiment 1, the physical condition diagnoser 13 determines the stomach health level based on the ratio between the frequency corresponding to the peak value of the frequency domain data and an ideal value that is the median of the target range. The stomach health level is the highest for the frequency corresponding to the peak value of the frequency domain data matching the ideal value. The stomach health level is lower for the frequency corresponding to the peak value of the frequency domain data deviating further from the ideal value.

The metabolism of an organ is closely related to heat production, and the heat production can be estimated based on the temperature variations in the organ. The physical condition diagnoser 13 thus determines the variations in the stomach metabolism based on the temperature variations in the stomach indicated by the activity-related body temperature data. The physical condition diagnoser 13 transmits, to the output device 53, the diagnosis results including the physical condition index indicating the stomach health level and the variations in the stomach metabolism. After the processing in step S13 in FIG. 3 is complete, the physical condition diagnosis device 1 ends the physical condition diagnosis process. The physical condition diagnosis device 1 repeats the above process at defined intervals.

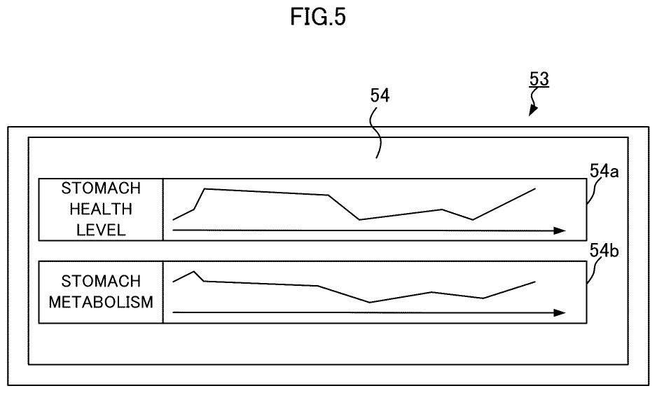

The output device 53 outputs at least one of visual or audio information indicating the physical condition index included in the received diagnosis results or a control signal corresponding to the physical condition index for an external device. For example, the output device 53 is a display device including a screen 54 to display the diagnosis results as illustrated in FIG. 5. The screen 54 includes a display area 54a to display the stomach health level included in the diagnosis results, and a display area 54b to display the variations in the stomach metabolism included in the diagnosis results.

As described above, the physical condition diagnosis device 1 according to Embodiment 1 determines the physical condition index of the subject organism based on the activity-related body temperature data indicating the temperature variations in a part of the subject organism caused by the activity of the subject organism. The physical condition index of the subject organism can thus be determined for physical condition diagnosis based on the temperature variations in the part of the subject organism before an abnormality in the body of the subject organism causes major variations in the body temperature as the dominant factor in the variations. The physical condition diagnosis device 1 according to Embodiment 1 can thus diagnose the physical condition of the subject organism accurately.

### Embodiment 2

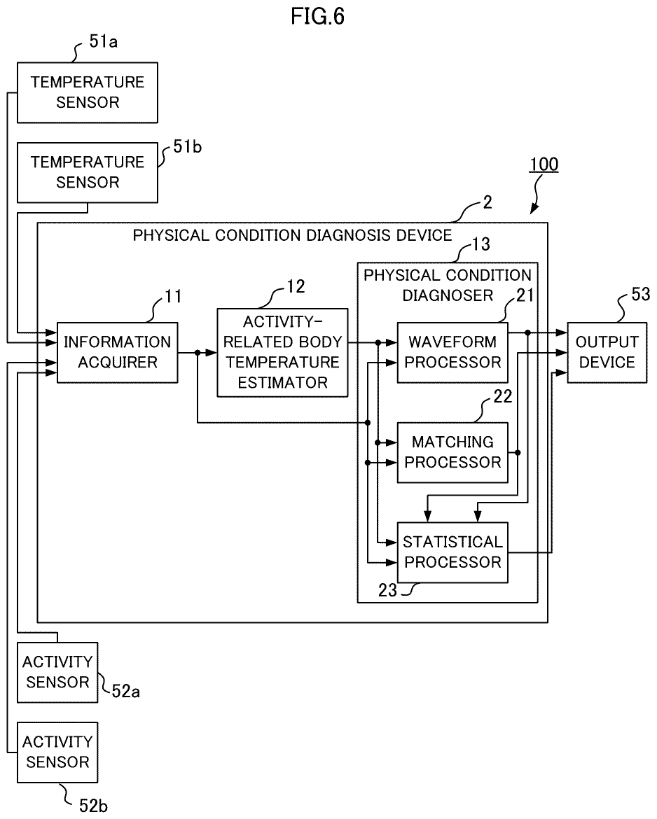

The physical condition index may be determined differently from the above example. The physical condition index may be determined based on vital sign information including temperature information of multiple parts and based on multiple types of activity information. A physical condition diagnosis device 2 according to Embodiment 2 illustrated in FIG. 6 determines the physical condition index indicating the physical condition of the subject organism based on the measurement results acquired by multiple temperature sensors 51a and 51b and the activity indexes generated by multiple activity sensors 52a and 52b.

The physical condition diagnoser 13 included in the physical condition diagnosis device 2 includes a matching processor 22 and a statistical processor 23, in addition to the waveform processor 21. The matching processor 22 performs pattern matching between the activity-related body temperature data and defined pattern data to determine the physical condition index. The statistical processor 23 determines another physical condition index of the subject organism based on at least one of the history of the activity-related body temperature data or a combination of physical condition indexes determined by the waveform processor 21 and the matching processor 22.

The physical condition diagnosis device 2 according to Embodiment 2 has a hardware configuration similar to the hardware configuration of the physical condition diagnosis device 1 according to Embodiment 1. However, the physical condition diagnosis device 2 is connected to the temperature sensors 51a and 51b, the activity sensors 52a and 52b, and the output device 53 through the interface 63.

The information acquirer 11 included in the physical condition diagnosis device 2 acquires the measurement results from the temperature sensors 51a and 51b as the temperature information. The temperature sensor 51a is attached to an armpit of the subject organism, similarly to the temperature sensor 51 in Embodiment 1. The temperature sensor 51b is attached to a calf surface of the subject organism.

The information acquirer 11 acquires, as the activity information, the activity indexes generated by the activity sensors 52a and 52b and type information indicating the types of activities associated with the activity sensors 52a and 52b. The activity sensor 52a includes a camera that captures an image of a meal for physical condition diagnosis, and a device that estimates the calorie intake from the captured meal. The activity sensor 52a outputs the estimated calorie intake as the activity index. When the estimated calorie intake is greater than or equal to a threshold intake, the activity sensor 52a outputs, as the type information, a signal at an H level indicating that the subject organism is eating. When the estimated calorie intake is less than the threshold intake, the activity sensor 52a outputs, as the type information, a signal at an L level indicating that the subject organism is not eating. The threshold intake refers to an intake indicating that the subject organism to undergo physical condition diagnosis is eating.

The activity sensor 52b is a speed sensor attached to the subject organism to measure the speed of the subject organism. For example, the activity sensor 52b is a wearable terminal wearable by the subject organism to undergo physical condition diagnosis and measures the speed of the subject organism. When the measured speed of the subject organism is within a target walking speed range, the activity sensor 52b outputs, as the type information, a signal at an H level indicating that the subject organism is walking. When the measured speed of the subject organism is out of the target walking speed range, the activity sensor 52b outputs, as the type information, a signal at an L level indicating that the subject organism is not walking. The target walking speed range refers to a range indicating that the subject organism is walking. The activity sensor 52b outputs the measured speed of the subject organism as the activity index.

After being activated, the physical condition diagnosis device 2 with the above structure repeats the process of physical condition diagnosis illustrated in FIG. 3 at defined intervals, as in Embodiment 1. The information acquirer 11 included in the physical condition diagnosis device 2 acquires the vital sign information and the variation factor information. The vital sign information includes the temperature information indicating the measurement results acquired by the temperature sensors 51a and 51b. The variation factor information includes the activity information including the type information and the activity indexes generated by the activity sensors 52a and 52b. The information acquirer 11 transmits the temperature information and the activity information to the activity-related body temperature estimator 12 and the physical condition diagnoser 13.

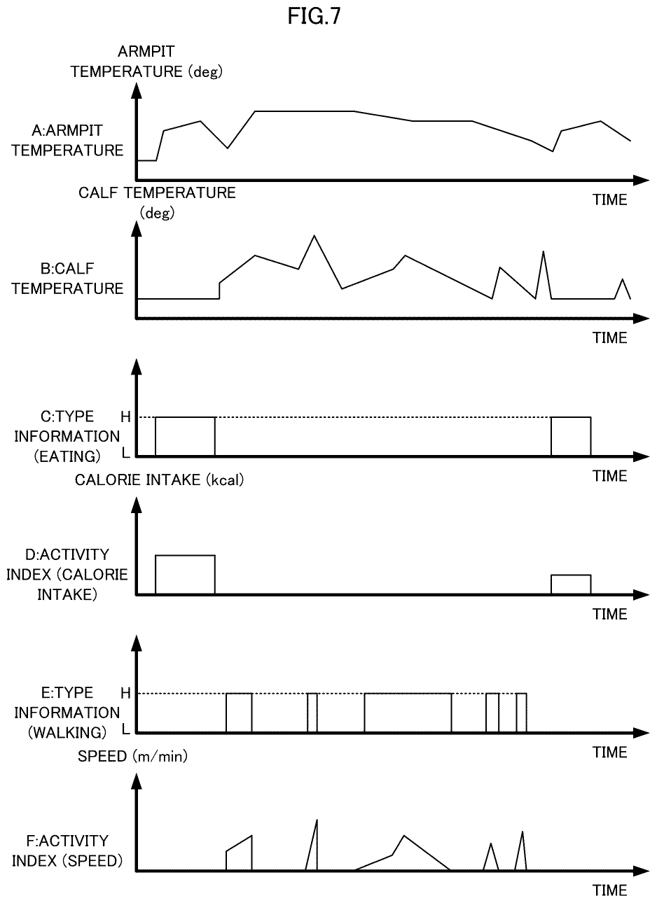

Graph A and graph B in FIG. 7 each illustrate an example of the temperature information included in the vital sign information acquired by the information acquirer 11. Graph A illustrates the temperature information output from the temperature sensor 51a. In graph A, the horizontal axis indicates time, and the vertical axis indicates armpit temperature (in degrees). Graph B illustrates the temperature information output from the temperature sensor 51b. In graph B, the horizontal axis indicates time, and the vertical axis indicates calf surface temperature (in degrees).

Graph C and graph D in FIG. 7 each illustrate an example of the activity information included in the variation factor information acquired by the information acquirer 11. Graph C illustrates the type information output from the activity sensor 52a. In graph C, the horizontal axis indicates time, and the vertical axis indicates the level of H or L. Graph D illustrates the activity index output from the activity sensor 52a. In graph D, the horizontal axis indicates time, and the vertical axis indicates calorie intake (in kcal).

Graph E and graph F in FIG. 7 each illustrate an example of the activity information included in the variation factor information acquired by the information acquirer 11. Graph E illustrates the type information output from the activity sensor 52b. In graph E, the horizontal axis indicates time, and the vertical axis indicates the level of H or L. Graph F illustrates the activity index output from the activity sensor 52b. In graph F, the horizontal axis indicates time, and the vertical axis indicates the speed of the subject organism (in m/min).

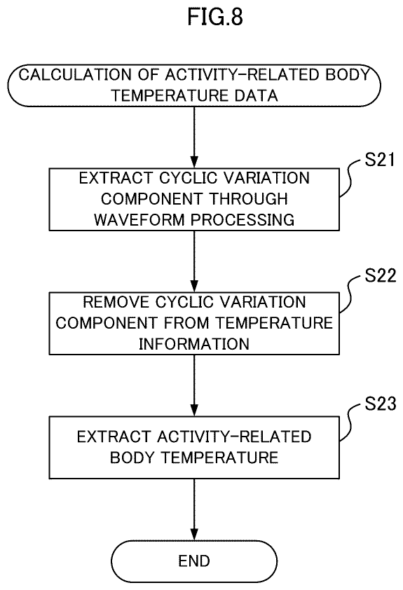

The activity-related body temperature estimator 12 determines, based on the vital sign information and the variation factor information, the activity-related body temperature data indicating the temperature variations in a part of the subject organism caused by the activity of the subject organism. More specifically, the activity-related body temperature estimator 12 determines the activity-related body temperature data through the process of calculating the activity-related body temperature data illustrated in FIG. 8. More specifically, the activity-related body temperature estimator 12 performs the process of calculating the activity-related body temperature data illustrated in FIG. 8 each time the temperature information and the activity information are acquired from the information acquirer 11.

The activity-related body temperature estimator 12 performs the FFT on each of the temperature information acquired from the temperature sensor 51a and the temperature information acquired from the temperature sensor 51b to generate frequency domain data. The activity-related body temperature estimator 12 extracts, from the frequency domain data, the frequency component corresponding to cyclic variations in the subject organism. The activity-related body temperature estimator 12 performs the inverse FFT (IFFT) on the frequency component to extract cyclic variation data in the time domain (step S21).

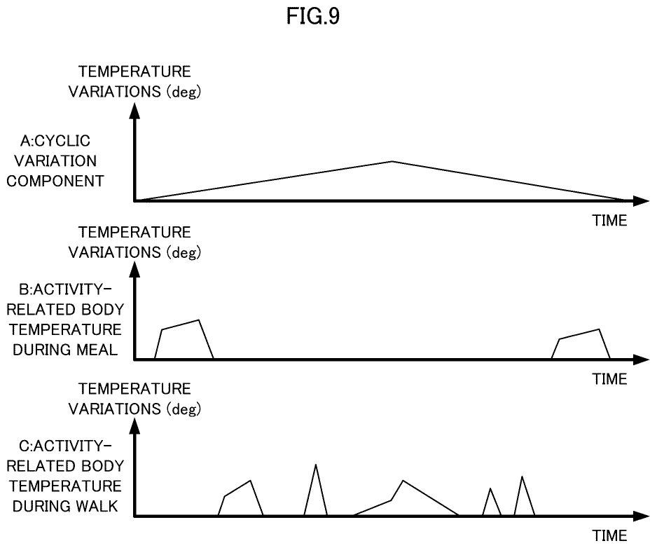

For example, the activity-related body temperature estimator 12 performs the FFT on each of the armpit temperature data indicated by the temperature information acquired from the temperature sensor 51a and the calf surface temperature data indicated by the temperature information acquired from the temperature sensor 51b to generate frequency domain data. The activity-related body temperature estimator 12 extracts the frequency component corresponding to cyclic variations of, for example, a circadian or ultradian rhythm from the frequency domain data. Graph A in FIG. 9 illustrates an example of the activity-related body temperature data indicating cyclic variations. In graph A, the horizontal axis indicates time, and the vertical axis indicates temperature variations (in degrees).

For example, with the human circadian rhythm being about 24 hours, the frequency component corresponding to the circadian rhythm can be acquired by extracting, from the frequency domain data, the frequency component corresponding to a cycle of 23 to 25 hours inclusive in the time domain. Thus, the activity-related body temperature estimator 12 performs, for example, the FFT on the armpit temperature data indicated by the temperature information acquired from the temperature sensor 51a to generate frequency domain data, and performs filtering using a low-pass filter (LPF) on the frequency domain data. Such filtering can extract the frequency component corresponding to the circadian rhythm. The activity-related body temperature estimator 12 performs the IFFT on the extracted frequency component to generate cyclic variation data in the time domain.

As illustrated in FIG. 8, the activity-related body temperature estimator 12 removes the cyclic variation component from the temperature information acquired from the temperature sensors 51a and 51b (step S22). More specifically, the activity-related body temperature estimator 12 performs filtering using a high-pass filter (HPF) on the frequency domain data generated in step S21 to remove the cyclic variation component corresponding to the circadian rhythm.

Using the data generated in step S22, the activity-related body temperature estimator 12 determines the activity-related body temperature data based on the activity information acquired from the activity sensors 52a and 52b (step S23).

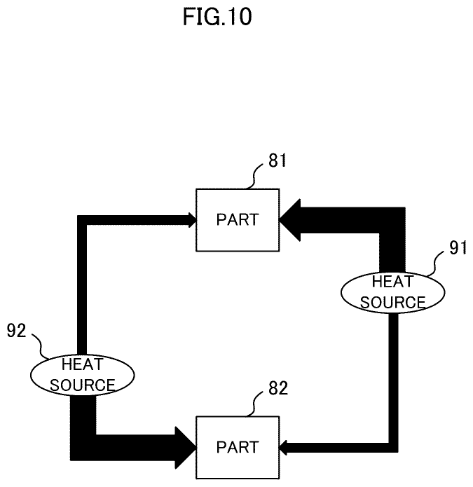

The activity-related body temperature estimator 12 estimates the stomach temperature of the subject organism based on the heat transfer path in the body of the subject organism as illustrated in FIG. 10, using the armpit temperature data resulting from the removal of the cyclic variation component in step S22 and the calf surface temperature data resulting from the removal of the cyclic variation component in step S22. In the body of the subject organism, heat is transferred through heat production and heat dissipation. Heat production includes, for example, heat generation from digestive organs during meals and heat generation from muscles during exercise. Heat dissipation includes, for example, sweating. The activity-related body temperature estimator 12 estimates the stomach temperature of the subject organism based on the heat transfer path in the body through such heat production and heat dissipation.

In the example in FIG. 10, heat is transferred from heat sources 91 and 92 to parts 81 and 82. The heat source 91 is, for example, the stomach that is active and generates heat while the subject organism is eating. The heat source 92 is, for example, a calf muscle that generates heat while the subject organism is exercising. The parts 81 and 82 are body parts of the subject organism to be measured with the temperature sensors 51a and 51b. The part 81 is, for example, an armpit. The part 82 is, for example, a calf surface.

In FIG. 10, the thickness of each arrow indicates the amount of transferred heat. The heat source 91 transfers more heat to the part 81 than to the part 82. The heat source 92 transfers more heat to the part 82 than to the part 81. In this case, a temperature change ΔT in the part 81 caused by heat production in the heat sources 91 and 92 can be expressed by a1×Δt1+a2×Δt2, where Δt1 is the temperature difference between the part 81 and the heat source 91, and Δt2 is the temperature difference between the part 81 and the heat source 92. The values a1 and a2 are defined coefficients. With the part 81 more strongly affected by the heat source 91, a1>a2. For example, a1=0.6 and a2=0.1. Using the above formula, the temperatures of the heat sources 91 and 92 can be estimated from the temperatures measured in the parts 81 and 82.

The activity-related body temperature estimator 12 estimates the stomach temperature of the subject organism based on the heat transfer path described above, using the armpit temperature data resulting from the removal of the cyclic variation component in step S22 in FIG. 8 and the calf surface temperature data resulting from the removal of the cyclic variation component in step S22. The activity-related body temperature estimator 12 determines, based on the estimated stomach temperature, the activity-related body temperature data indicating the temperature variations in the stomach for the period in which the type information generated by the activity sensor 52a indicates an H level.

Graph B in FIG. 9 illustrates an example of the activity-related body temperature data indicating the temperature variations in the stomach. In graph B, the horizontal axis indicates time, and the vertical axis indicates temperature variations (in degrees).

The activity-related body temperature estimator 12 also determines, using the calf surface temperature data resulting from the removal of the cyclic variation component in step S22, the temperature variations in the calf muscle for the period in which the type information included in the activity information acquired from the activity sensor 52b indicates an H level.

Graph C in FIG. 9 illustrates an example of the activity-related body temperature data indicating the temperature variations in the calf muscle. In graph C, the horizontal axis indicates time, and the vertical axis indicates temperature variations (in degrees).

After determining the activity-related body temperature data for each part as described above, the activity-related body temperature estimator 12 transmits the determined activity-related body temperature data to the physical condition diagnoser 13, and ends the process of calculating the activity-related body temperature data.

The waveform processor 21 included in the physical condition diagnoser 13 determines the stomach health level and the variations in the stomach metabolism, as in Embodiment 1. The waveform processor 21 acquires the temperature information from the information acquirer 11, and outputs the diagnosis results to the statistical processor 23 and the output device 53. The diagnosis results include the temperature information, such as information about the armpit temperature of the subject organism, the stomach health level, and the variations in the stomach metabolism.

The matching processor 22 performs pattern matching between the activity-related body temperature data indicating the temperature variations in a part of the subject organism caused by exercise and defined pattern data to diagnose the physical condition of the subject organism. More specifically, the matching processor 22 performs pattern matching between the activity-related body temperature data indicating the temperature variations in the calf muscle determined by the activity-related body temperature estimator 12 and defined pattern data indicating the temperature variations during a spasm.

The matching processor 22 determines the spasm severity as the physical condition index based on the degree of match between the activity-related body temperature data indicating the temperature variations in the calf muscle and the defined pattern data. A high degree of match between the activity-related body temperature data indicating the temperature variations in the calf muscle and the pattern data indicates a high spasm severity. A sufficiently low degree of match between the activity-related body temperature data indicating the temperature variations in the calf muscle and the pattern data indicates a low spasm severity or substantially no spasm. The matching processor 22 outputs the diagnosis results including the physical condition index indicating the spasm severity to the statistical processor 23 and the output device 53.

The statistical processor 23 acquires the activity-related body temperature data from the activity-related body temperature estimator 12, and acquires the diagnosis results including the physical condition indexes from the waveform processor 21 and the matching processor 22. The statistical processor 23 determines another physical condition index based on at least one of the history of the activity-related body temperature data or a combination of the physical condition indexes determined by the waveform processor 21 and the matching processor 22.

The statistical processor 23 stores the activity-related body temperature data each time the activity-related body temperature data is acquired from the activity-related body temperature estimator 12. The statistical processor 23 thus generates the history of the activity-related body temperature data. The statistical processor 23 performs statistical processing on the history of the activity-related body temperature data to determine whether the history has a pattern with concerns, such as whether the stomach temperature increases markedly during digestion on a regular basis. The statistical processor 23 can thus determine the physical condition index indicating whether the stomach has a chronic disease.

The statistical processor 23 determines another physical condition index, such as a physical condition index indicating whether the subject organism is in a healthy condition or the health level of the subject organism, based on a combination of the physical condition indexes determined by the waveform processor 21 and the matching processor 22. The statistical processor 23 outputs the diagnosis results including the determined physical condition index to the output device 53.

In an example, the statistical processor 23 determines that the subject organism is in a fine physical condition and is healthy both when the stomach health level determined by the waveform processor 21 is greater than or equal to a first threshold and when the spasm severity determined by the matching processor 22 is less than or equal to a second threshold. The statistical processor 23 determines that the subject organism is in a poor physical condition and is unhealthy when the stomach health level determined by the waveform processor 21 is lower than the first threshold or when the spasm severity determined by the matching processor 22 is higher than the second threshold. The first threshold is, for example, the median of the possible range of health levels. The second threshold is, for example, the median of the possible range of spasm severities.

The statistical processor 23 also determines the health level of the subject organism based on the physical condition indexes determined by the waveform processor 21 and the matching processor 22. The health level is an index that is the highest both when the stomach health level determined by the waveform processor 21 is at the upper limit and when the spasm severity determined by the matching processor 22 is at the lower limit, and is the lowest both when the stomach health level determined by the waveform processor 21 is at the lower limit and when the spasm severity determined by the matching processor 22 is at the upper limit.

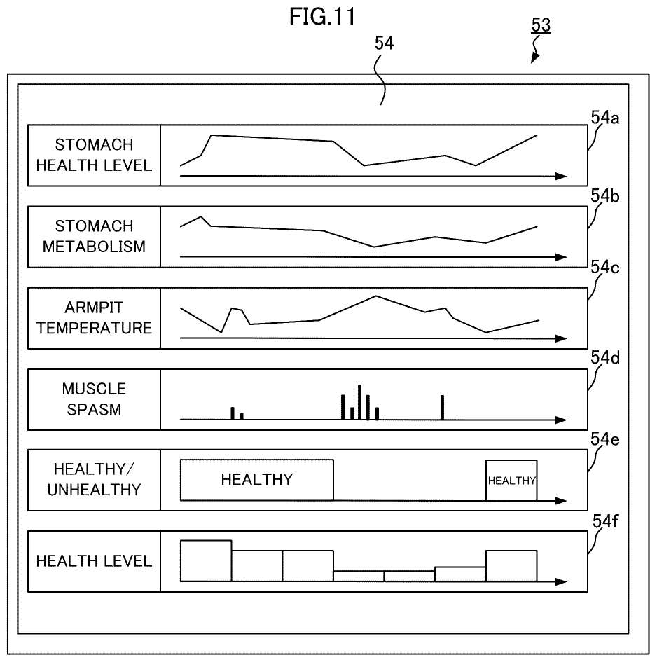

The output device 53 outputs at least one of visual or audio information indicating the diagnosis results received from the physical condition diagnoser 13 or a control signal corresponding to the diagnosis results for an external device. For example, the output device 53 is a display device including the screen 54 to display the diagnosis results, as illustrated in FIG. 11. The screen 54 includes the display area 54a to display the stomach health level included in the diagnosis results acquired by the waveform processor 21, and the display area 54b to display the variations in the stomach metabolism included in the diagnosis results acquired by the waveform processor 21. The screen 54 includes a display area 54c to display the temperature information included in the diagnosis results acquired by the waveform processor 21, or specifically, the armpit temperature.

The screen 54 includes a display area 54d to display the spasm severity included in the diagnosis results acquired by the matching processor 22. The screen 54 includes a display area 54e to display the determination as to whether the subject organism is in a healthy condition included in the diagnosis results acquired by the statistical processor 23. The screen 54 includes a display area 54f to display the health level of the subject organism included in the diagnosis results acquired by the statistical processor 23.

As described above, each of the waveform processor 21 and the matching processor 22 included in the physical condition diagnoser 13 in the physical condition diagnosis device 2 according to Embodiment 2 determines the physical condition index. The statistical processor 23 included in the physical condition diagnosis device 2 determines another physical condition index based on at least one of an activity-related body temperature in the past or a combination of the physical condition indexes determined by the waveform processor 21 and the matching processor 22. The physical condition diagnosis device 2 can diagnose the physical condition of the subject organism by determining multiple physical condition indexes. The physical condition diagnosis device 2 can thus diagnose the physical condition of the subject organism accurately.

The statistical processor 23 can diagnose the physical condition based on diagnosis results in the past. This reduces the likelihood that the physical condition of the subject organism is diagnosed erroneously based on temporary variations in the measurement results acquired by the temperature sensor 51a or 51b or the activity sensor 52a or 52b.

### Embodiment 3

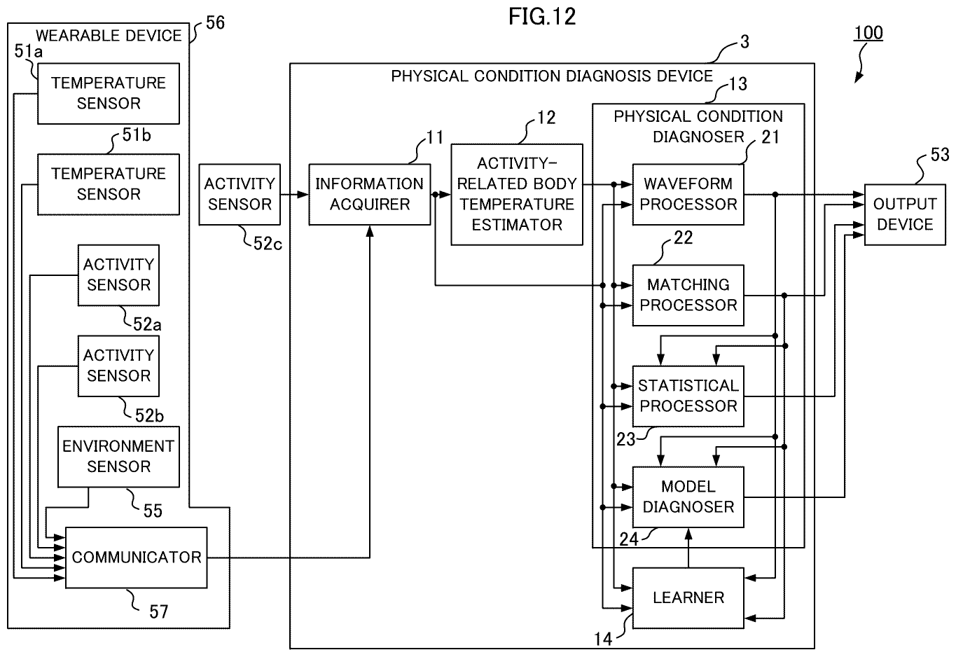

The physical condition index may be determined differently from the above examples. The physical condition index may be determined by inputting the activity-related body temperature data into a learning model. The activity-related body temperature data may be determined based on the variation factor information including environmental information indicating the environment surrounding the subject organism, in addition to including the activity information. A physical condition diagnosis device 3 according to Embodiment 3 illustrated in FIG. 12 determines the physical condition index indicating the physical condition of the subject organism based on the measurement results acquired by multiple temperature sensors 51a and 51b, the activity indexes generated by multiple activity sensors 52a, 52b, and 52c, and the measurement results acquired by an environment sensor 55.

The physical condition diagnosis device 3 includes the same components as the physical condition diagnosis device 2 according to Embodiment 2, and additionally includes a learner 14 that determines a physical condition model for deriving the physical condition index from the activity-related body temperature data and the variation factor information. The physical condition diagnoser 13 includes the waveform processor 21, the matching processor 22, the statistical processor 23, and a model diagnoser 24. The model diagnoser 24 inputs the activity-related body temperature data and the variation factor information into the physical condition model determined by the learner 14 to determine the physical condition index of the subject organism.

The physical condition diagnosis device 3 according to Embodiment 3 has a hardware configuration similar to the hardware configuration of the physical condition diagnosis device 1 according to Embodiment 1. However, the physical condition diagnosis device 3 is connected to the temperature sensors 51a and 51b, the activity sensors 52a, 52b, and 52c, the environment sensor 55, and the output device 53 through the interface 63.

The information acquirer 11 included in the physical condition diagnosis device 3 acquires the measurement results from the temperature sensors 51a and 51b as the temperature information, as in Embodiment 2. The information acquirer 11 acquires, as the activity information, the activity indexes generated by the activity sensors 52a and 52b and the type information indicating the types of activities associated with the activity sensors 52a and 52b, as in Embodiment 2. The information acquirer 11 acquires, from the activity sensor 52c, the activity index indicating the measurement value of a water intake. The activity sensor 52c is attached to a bottle containing drinking water to measure the water intake of the subject organism to undergo physical condition diagnosis.

The information acquirer 11 acquires, as the environmental information, the measurement results of physical quantities indicating the environment surrounding the subject organism from the environment sensor 55. The environment sensor 55 measures, for example, brightness, ambient temperature, humidity, odor, gas concentration, soil humidity, atmospheric pressure, radiation intensity, magnetism, weather, dust, or infrared light. In Embodiment 3, the environment sensor 55 measures ambient temperature and humidity.

In Embodiment 3, the temperature sensors 51a and 51b, the activity sensors 52a and 52b, and the environment sensor 55 are implemented in a wearable device 56 wearable by the subject organism. In an example, the wearable device 56 may be a headset or smart glasses including the temperature sensors 51a and 51b, the activity sensors 52a and 52b, and the environment sensor 55. The information acquirer 11 acquires the temperature information, the activity information, and the environmental information from a communicator 57 included in the wearable device 56 worn on the subject organism. More specifically, the communicator 57 transmits the temperature information acquired from the temperature sensors 51a and 51b, the activity information acquired from the activity sensors 52a and 52b, and the environmental information acquired from the environment sensor 55 to the information acquirer 11 included in the physical condition diagnosis device 3.

The activity-related body temperature estimator 12 estimates the temperature of a part of the subject organism, in addition to calculating the activity-related body temperature data. For example, the activity-related body temperature estimator 12 estimates the brain temperature of the subject organism based on the temperature information, the activity information, and the environmental information. More specifically, the temperature information indicates the armpit temperature and the calf surface temperature. The activity information indicates the calorie intake, the moving speed, and the water intake. The environmental information indicates the ambient temperature and the humidity. The activity-related body temperature estimator 12 transmits, to the physical condition diagnoser 13, the activity-related body temperature data and estimated data indicating the estimated temperatures.

The waveform processor 21 included in the physical condition diagnoser 13 determines the stomach health level and the variations in the stomach metabolism, as in Embodiment 1. The waveform processor 21 acquires the temperature information from the information acquirer 11, and acquires the activity-related body temperature data and the estimated data from the activity-related body temperature estimator 12. The waveform processor 21 outputs, to the statistical processor 23 and the output device 53, the diagnosis results including the temperature information (e.g., information about the armpit temperature of the subject organism), the stomach health level, the variations in the stomach metabolism, and the estimated data (e.g., the estimated brain temperature).

The matching processor 22 acquires the temperature information from the information acquirer 11, and acquires the activity-related body temperature data from the activity-related body temperature estimator 12. The matching processor 22 determines the spasm severity, as in Embodiment 2. The matching processor 22 also determines the risk of dehydration by pattern matching between a data pattern corresponding to the ambient temperature and the humidity included in the environmental information and the pattern of the armpit temperature included in the temperature information. The matching processor 22 adjusts the risk of dehydration based on at least one of the intensity of exercise estimated from the speed of the subject organism indicated by the activity sensor 52b or the water intake of the subject organism acquired from the activity sensor 52c. For example, exercising intensely in a hot environment increases the risk of dehydration, whereas drinking water in a cool environment reduces the risk of dehydration.

The matching processor 22 also determines whether the symptoms of heatstroke are occurring by pattern matching between the data pattern corresponding to the ambient temperature and the humidity included in the environmental information and the pattern of the armpit temperature included in the temperature information. The matching processor 22 determines the risk of heatstroke based on the risk of dehydration, the armpit temperature included in the temperature information, and the water intake of the subject organism acquired from the activity sensor 52c. The matching processor 22 transmits, to the statistical processor 23 and the output device 53, the diagnosis results including data of the water intake and the physical condition index indicating the risk of dehydration, whether the symptoms of heatstroke are occurring, and the risk of heatstroke.

The learner 14 acquires the variation factor information from the information acquirer 11, and acquires the activity-related body temperature data from the activity-related body temperature estimator 12. The learner 14 acquires the physical condition index from each of the waveform processor 21 and the matching processor 22. The learner 14 learns the association between the activity-related body temperature data, the variation factor information, and each physical condition index.

The learner 14 determines a physical condition model that is a neural network model for deriving the physical condition index of the subject organism from the activity-related body temperature data and the variation factor information. More specifically, the learner 14 learns first training data including the activity-related body temperature data, the variation factor information, and the physical condition index determined by the waveform processor 21. The learner 14 generates a first physical condition model that is a neural network model including an input layer, intermediate layers, and an output layer. The first physical condition model receives the activity-related body temperature data and the variation factor information as input values, and outputs the physical condition index determined by the waveform processor 21, or specifically, the stomach health level and the variations in the stomach metabolism as output values. The first physical condition model is, for example, a recurrent neural network (RNN), a long short-term memory (LSTM), or a general-purpose artificial intelligence model. For the first physical condition model, the learner 14 adjusts, based on the first training data, weights between the input layer and the intermediate layer, weights between the intermediate layers, and weights between the intermediate layer and the output layer.

The learner 14 learns second training data including the activity-related body temperature data, the variation factor information, and the physical condition index determined by the matching processor 22. The learner 14 generates a second physical condition model that is a neural network model including an input layer, intermediate layers, and an output layer. The second physical condition model receives the activity-related body temperature data and the variation factor information as input values, and outputs the physical condition index determined by the matching processor 22, or specifically, the spasm severity, whether the symptoms of heatstroke are occurring, and the risk of heatstroke as output values. For the second physical condition model, the learner 14 adjusts, based on the second training data, weights between the input layer and the intermediate layer, weights between the intermediate layers, and weights between the intermediate layer and the output layer.

The learner 14 learns third training data including the activity-related body temperature data, the variation factor information, at least one of the physical condition index determined by the waveform processor 21 or the physical condition index determined by the matching processor 22, and the physical condition index determined by the statistical processor 23. The learner 14 generates a third physical condition model that is a neural network model including an input layer, intermediate layers, and an output layer. The third physical condition model receives the activity-related body temperature data, the variation factor information, and at least one of the physical condition index determined by the waveform processor 21 or the physical condition index determined by the matching processor 22 as input values, and outputs the physical condition index determined by the statistical processor 23, or specifically, whether the subject organism is in a healthy condition and the health level as output values. For the third physical condition model, the learner 14 adjusts, based on the third training data, weights between the input layer and the intermediate layer, weights between the intermediate layers, and weights between the intermediate layer and the output layer.

The learner 14 transmits a physical condition model including at least one of the first physical condition model, the second physical condition model, or the third physical condition model to the model diagnoser 24.

The model diagnoser 24 inputs the variation factor information acquired from the information acquirer 11 and the activity-related body temperature data acquired from the activity-related body temperature estimator 12 into the physical condition model acquired from the learner 14 to determine the physical condition index of the subject organism. More specifically, the model diagnoser 24 inputs the activity-related body temperature data and the variation factor information into the first physical condition model to determine the physical condition index, or specifically, the stomach health level and the variations in the stomach metabolism.

The model diagnoser 24 inputs the activity-related body temperature data and the variation factor information into the second physical condition model to determine the physical condition index, or specifically, the spasm severity, whether the symptoms of heatstroke are occurring, and the risk of heatstroke.

The model diagnoser 24 inputs the activity-related body temperature data, the variation factor information, and the physical condition indexes determined by the waveform processor 21 and the matching processor 22 into the third physical condition model to determine the physical condition index, or specifically, whether the subject organism is in a healthy condition and the health level.

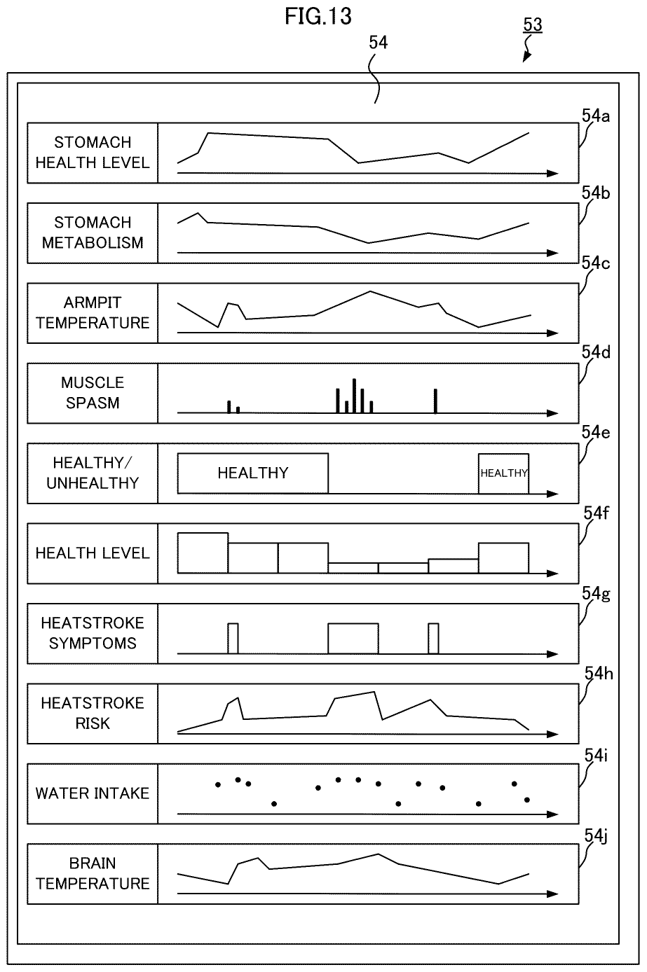

The output device 53 outputs at least one of visual or audio information indicating the diagnosis results received from the physical condition diagnoser 13 or a control signal corresponding to the diagnosis results for an external device. For example, the output device 53 is a display device including the screen 54 to display the diagnosis results, as illustrated in FIG. 13. As in Embodiment 1, the screen 54 includes the display area 54a to display the stomach health level included in the diagnosis results acquired by the waveform processor 21, and the display area 54b to display the variations in the stomach metabolism included in the diagnosis results acquired by the waveform processor 21.

As in Embodiment 2, the screen 54 includes the display area 54c to display the temperature information included in the diagnosis results acquired by the waveform processor 21, or specifically, the armpit temperature. The screen 54 includes the display area 54d to display the spasm severity included in the diagnosis results acquired by the matching processor 22. The screen 54 includes the display area 54e to display the determination as to whether the subject organism is in a healthy condition included in the diagnosis results acquired by the statistical processor 23. The screen 54 includes the display area 54f to display the health level of the subject organism included in the diagnosis results acquired by the statistical processor 23.

The screen 54 includes a display area 54g to display whether the symptoms of heatstroke are occurring included in the diagnosis results acquired by the matching processor 22. The screen 54 includes a display area 54h to display the risk of heatstroke included in the diagnosis results acquired by the matching processor 22. The screen 54 includes a display area 54i to display the water intake included in the diagnosis results acquired by the matching processor 22. The screen 54 includes a display area 54j to display the estimated data about the brain temperature included in the diagnosis results acquired by the waveform processor 21.

As described above, the model diagnoser 24 included in the physical condition diagnosis device 3 according to Embodiment 3 inputs the activity-related body temperature data and the variation factor information or inputs the activity-related body temperature data, the variation factor information, and the physical condition index into the physical condition model as a neural network model to determine another physical condition index. This allows accurate diagnosis of the physical condition of the subject organism.

The present disclosure is not limited to the above embodiments. The embodiments may be combined as appropriate. In an example, each of the physical condition diagnosis device 1 according to Embodiment 1 and the physical condition diagnosis device 2 according to Embodiment 2 may include the learner 14 as in Embodiment 3.

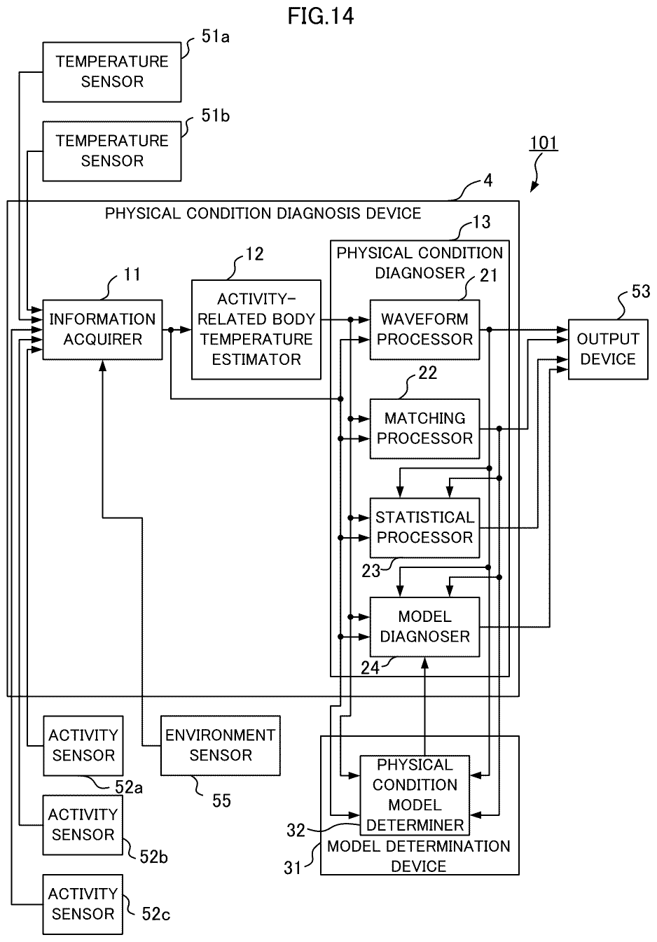

The physical condition diagnosis device may acquire the physical condition model from an external device. A physical condition diagnosis system 101 illustrated in FIG. 14 includes the temperature sensors 51a and 51b, the activity sensors 52a, 52b, and 52c, the environment sensor 55, a physical condition diagnosis device 4, the output device 53, and a model determination device 31. The model determination device 31 determines the physical condition model to be used for physical condition diagnosis. The physical condition diagnosis device 4 has the same structure as the physical condition diagnosis device 3, but without the learner 14. The model determination device 31 includes a physical condition model determiner 32 that performs the same process as the learner 14 included in the physical condition diagnosis device 3 according to Embodiment 3.

The physical condition model determiner 32 acquires the variation factor information from the information acquirer 11, acquires the activity-related body temperature data from the activity-related body temperature estimator 12, and acquires the physical condition indexes from the waveform processor 21, the matching processor 22, and the statistical processor 23 included in the physical condition diagnoser 13. Similarly to the learner 14 included in the physical condition diagnosis device 3 according to Embodiment 3, the physical condition model determiner 32 learns the association between the activity-related body temperature data, the variation factor information, and each physical condition index indicating the physical condition of the subject organism, and determines the physical condition model for deriving the physical condition index from the activity-related body temperature data and the variation factor information. The physical condition model determiner 32 transmits the determined physical condition model to the model diagnoser 24 included in the physical condition diagnoser 13.

The model diagnoser 24 acquires the physical condition model from the physical condition model determiner 32. As in Embodiment 3, the model diagnoser 24 inputs the variation factor information acquired from the information acquirer 11 and the activity-related body temperature data acquired from the activity-related body temperature estimator 12 into the physical condition model acquired from the physical condition model determiner 32 to determine the physical condition index of the subject organism.

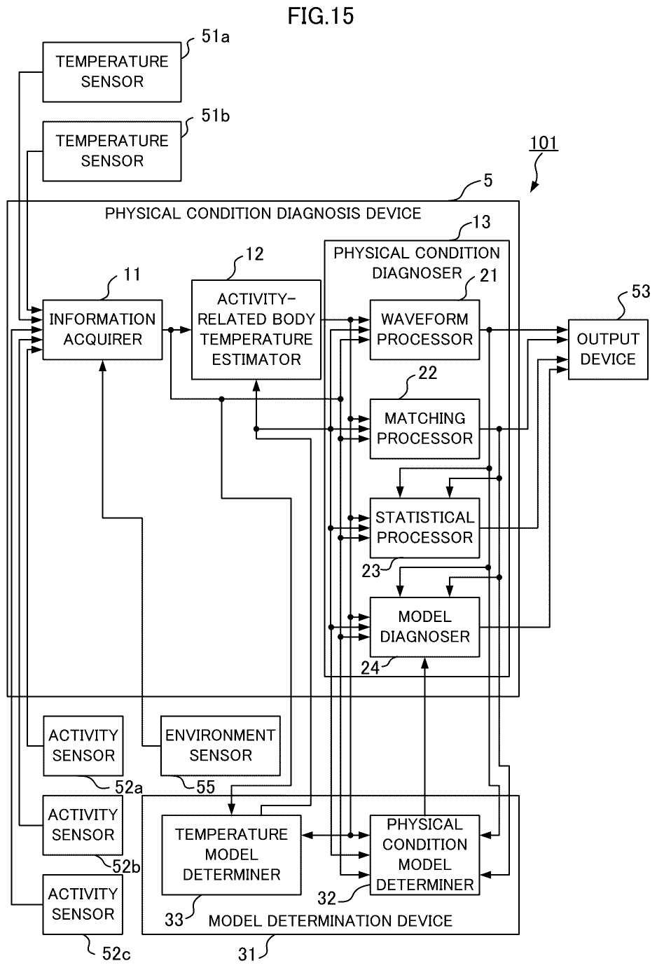

The model determination device 31 may determine a temperature model for deriving the activity-related body temperature data from the vital sign information and the variation factor information, in addition to determining the physical condition model. A physical condition diagnosis system 101 illustrated in FIG. 15 includes the temperature sensors 51a and 51b, the activity sensors 52a, 52b, and 52c, the environment sensor 55, a physical condition diagnosis device 5, the output device 53, and the model determination device 31. The model determination device 31 determines the physical condition model to be used for physical condition diagnosis and the temperature model for deriving the activity-related body temperature data. The physical condition diagnosis device 5 has the same structure as the physical condition diagnosis device 3, but without the learner 14.

A temperature model determiner 33 acquires the vital sign information and the variation factor information from the information acquirer 11, and acquires the activity-related body temperature data from the activity-related body temperature estimator 12. The temperature model determiner 33 learns the association between the vital sign information, the variation factor information, and the activity-related body temperature data. The temperature model determiner 33 determines the temperature model that is a neural network model that receives inputs of the vital sign information and the variation factor information and outputs the activity-related body temperature data. More specifically, the temperature model determiner 33 learns temperature training data including the vital sign information, the variation factor information, and the activity-related body temperature data. The temperature model determiner 33 generates the temperature model that is a neural network model including an input layer, intermediate layers, and an output layer. The temperature model receives the vital sign information and the variation factor information as input values, and outputs the activity-related body temperature data as an output value. For the temperature model, the temperature model determiner 33 adjusts, based on the temperature training data, weights between the input layer and the intermediate layer, weights in the intermediate layers, and weights in the intermediate layers and the output layer. The temperature model determiner 33 transmits the determined temperature model to the activity-related body temperature estimator 12.

The activity-related body temperature estimator 12 acquires the temperature model from the temperature model determiner 33. The activity-related body temperature estimator 12 inputs the vital sign information and the variation factor information acquired from the information acquirer 11 into the temperature model acquired from the temperature model determiner 33 to determine the activity-related body temperature data. The activity-related body temperature estimator 12 transmits the determined activity-related body temperature data to the physical condition diagnoser 13.

The learner 14 included in the physical condition diagnosis device 3 may determine the temperature model and determine the activity-related body temperature data based on the determined temperature model, similarly to the temperature model determiner 33. In this case, the activity-related body temperature estimator 12 inputs the vital sign information and the variation factor information acquired from the information acquirer 11 into the temperature model acquired from the learner 14 to determine the activity-related body temperature data.

The temperature model may be a neural network model that receives inputs of the vital sign information, the variation factor information, and the environmental information and outputs the activity-related body temperature data.

The subject organism may be any organism, such as a human, a livestock animal, or a pet. The activity of the subject organism may be any activity that causes temperature variations in a part of the subject organism, including an activity that causes a decrease in the body temperature such as sleep and meditation, as well as an activity that causes heat production such as eating and exercise.

The information acquirer 11 may acquire the vital sign information based on data input into each of the physical condition diagnosis devices 1 to 5. In an example, the information acquirer 11 may acquire information about the blood sugar level of the subject organism as the vital sign information. When the blood sugar level varies in cycles shorter than eight hours, such variations can be attributed to eating and digestion. In this case, the physical condition diagnoser 13 can determine whether nourishment is absorbed appropriately based on, for example, the variations in the blood sugar level and the variations in the activity-related body temperature corresponding to the stomach temperature.

The information acquirer 11 may acquire the activity information from data input into each of the physical condition diagnosis devices 1 to 5. In an example, the information acquirer 11 may acquire the type information indicating the activity state based on a predetermined action of the subject organism.

The information acquirer 11 may acquire the measurement results from a temperature sensor installed near an artery and a temperature sensor installed near a vein. A temperature change upstream of the blood flow appears as the phase difference between the measurement results acquired by the temperature sensor installed near the artery and the measurement results acquired by the temperature sensor installed near the vein. In this case, the activity-related body temperature estimator 12 may determine the activity-related body temperature data based on the phase difference. More specifically, the activity-related body temperature estimator 12 may remove, from the temperature information, a component of the activity-related body temperature data determined based on the phase difference, thus generating temperature information independent of the blood flow.

The information acquirer 11 may acquire an index of exercise intensity as the activity information. For a healthy subject organism, the amplitude of the activity-related body temperature data is greater at higher exercise intensities and is smaller at lower exercise intensities. The physical condition diagnoser 13 may determine the physical condition index of the subject organism, such as the health level, based on whether the exercise intensity and the amplitude of the activity-related body temperature data have a correlation. More specifically, the physical condition diagnoser 13 may determine the health level of the subject organism based on, for example, the difference in phase, frequency spectrum, or amplitude between the waveform data of the index of exercise intensity and the waveform data of the activity-related body temperature data.

The activity-related body temperature estimator 12 may determine the activity-related body temperature data differently from the above example. In an example, the activity-related body temperature estimator 12 may compare the armpit temperature with a waveform pattern defined based on temperature variations during a meal, and extract, as the activity-related body temperature data, the waveform of the armpit temperature matching the waveform pattern.

The activity-related body temperature estimator 12 may extract the frequency component corresponding to the duration of an activity from frequency domain data, and perform the IFFT on the extracted frequency component to determine the activity-related body temperature data in the time domain.

The activity-related body temperature estimator 12 may remove the cyclic variation component by subtracting a reference waveform corresponding to the cyclic variation component from the waveform data of the temperature information. In another example, the activity-related body temperature estimator 12 may determine, based on the armpit temperature, the cyclic variation component corresponding to the circadian rhythm through cosinor analysis for determining parameters of an approximate cosine wave with the least squares method.

The activity-related body temperature estimator 12 may calculate, as the activity-related body temperature data, fluctuation of the immediately preceding measurement value from a reference value or fluctuation of the average of measurement values in the most recent specific period from the reference value, specifically, fluctuation of the armpit temperature from the reference value.

The heat transfer path illustrated in FIG. 10 is a mere example. A part of the subject organism near the body surface can be primarily affected by heat exchange with the outside of the subject organism. A part of the subject organism can be primarily affected by heat exchange with an adjacent organ. A part of the subject organism can be affected by an upstream portion adjacent to the heart at varying levels depending on the blood vessel thickness. The activity-related body temperature estimator 12 may estimate the activity-related body temperature based on such factors.

The physical condition diagnoser 13 is not limited to the above example. In an example, the physical condition diagnoser 13 may acquire multiple pieces of activity-related body temperature data different from each other in at least one of the target part or the variation factor, and may diagnose the physical condition of the subject organism based on the correlation between pieces of waveform data of the multiple pieces of activity-related body temperature data in the time domain.

The physical condition diagnoser 13 may output the diagnosis results including the vital sign information, the variation factor information including the activity information and the environmental information, at least one of the multiple pieces of activity-related body temperature data, and the physical condition index. In an example, the physical condition index determined by the waveform processor 21 can indicate a decrease in the liver function. In this case, the physical condition diagnoser 13 may output the diagnosis results including, for example, the physical condition index indicating the liver function, the activity-related body temperature data about the liver, the bladder, or other organs, and the estimated data indicating the temperature of the liver, the bladder, or other organs.

The waveform processor 21 may use frequency domain data resulting from the IFFT performed on the activity-related body temperature data, and diagnose the physical condition of the subject organism based on, for example, whether the frequency component corresponding to the peak value is within a reference range, whether the data includes a peak value, or whether a defined frequency component has an amplitude within a target range.

When the waveform data of the activity-related body temperature data has an amplitude out of the target amplitude range based on the identification information corresponding to the activity-related body temperature data, the waveform processor 21 may diagnose an abnormality in the organ corresponding to the activity.

The waveform processor 21 may diagnose the physical condition of the subject organism based on the activity-related body temperature data for any defined period, such as a specific period between 0:00 and 24:00, a period corresponding to the activity information, or the period from a rising edge to the next rising edge of the waveform data of the body temperature data.

The waveform processor 21 may acquire multiple types of activity-related body temperature data, and determine the physical condition index of the subject organism based on at least one of the phase, the amplitude, the frequency spectrum, or the waveform of the waveform data of each piece of activity-related body temperature data. In this case, the waveform processor 21 may output the diagnosis results including multiple physical condition indexes to the statistical processor 23. The statistical processor 23 may determine another physical condition index of the subject organism based on the multiple physical condition indexes determined by the waveform processor 21.

In an example, the waveform processor 21 may determine the physical condition index of the subject organism based on the frequency spectrum of the activity-related body temperature data for each of eating and excretion. In this case, the statistical processor 23 can determine, based on the physical condition index for each of eating and excretion, the physical condition index indicating the health level for an organ associated with both eating and excretion and for an organ associated with one of eating or excretion.

The matching processor 22 may determine the physical condition index based on the activity-related body temperature data and the variation factor information. In an example, the matching processor 22 may determine the spasm severity by pattern matching between the activity-related body temperature data indicating the temperature variations in the calf muscle determined by the activity-related body temperature estimator 12 and defined pattern data indicating the temperature variations during a spasm. The matching processor 22 may also determine the cause of the spasm based on the variation factor information. In an example, when the variation factor information indicates that exercise is not performed in the most recent period, the cause of the spasm can be determined to be a cramp due to a lack of exercise. In another example, when the variation factor information indicates that exercise is performed in the most recent period, the cause of the spasm can be determined to be dehydration. The matching processor 22 outputs, to the output device 53, the diagnosis results including the physical condition index indicating the spasm severity and the cause of the spasm.

The statistical processor 23 may combine physical condition indexes associated with organs to estimate a disease of the subject organism based on a combination of abnormalities in the organs.

The statistical processor 23 may determine the physical condition index by combining the activity-related body temperature data and the variation factor information. In an example, when the activity-related body temperature data has an upward trend in the most recent specific period and the variation factor information indicates no defecation in the most recent specific period, the statistical processor 23 may determine the physical condition index indicating constipation.

The learner 14 may generate the physical condition model differently from the above example. The learner 14 may learn, in a manner associated with one another, the activity-related body temperature information, the activity information, the physical condition index determined by the matching processor 22, and the physical condition index determined by the waveform processor 21. The learner 14 may determine the first physical condition model that receives inputs of the activity-related body temperature information, the activity information, and the physical condition index determined by the matching processor 22 and outputs the physical condition index determined by the waveform processor 21.

The learner 14 may perform supervised learning based on multidimensional function fitting to generate the physical condition model and the temperature model. Supervised learning refers to providing a large number of training datasets, or specifically, sets of input and output data, to a learning device to cause the learning device to learn features from the large number of datasets to generate a model for estimating the output from the input.

Similarly, the physical condition model determiner 32 and the temperature model determiner 33 may perform supervised learning based on multidimensional function fitting to respectively generate the physical condition model and the temperature model.

The diagnosis results transmitted from the physical condition diagnoser 13 to the output device 53 may include at least one of the vital sign information or the variation factor information, or may include the activity-related body temperature data, in addition to the physical condition index acquired by each component of the physical condition diagnoser 13. The variation factor information may include both the activity information and the environmental information, or may include one of the activity information or the environmental information. The activity information included in the variation factor information may include both the type information and the activity index, or may include one of the type information or the activity index.

The statistical processor 23 may store the physical condition index each time the physical condition index is acquired from the waveform processor 21 or the matching processor 22, and may determine another physical condition index of the subject organism based on a physical condition index determined in the past. In an example, the statistical processor 23 may determine that the subject organism is likely to have post-traumatic stress disorder (PTSD) both when the spasm severity determined by the matching processor 22 is high throughout the year without seasonal variation and when such a high spasm severity occurs at a frequency higher than or equal to a threshold. In another example, the statistical processor 23 may determine that the subject organism is likely to be sensitive to the cold rather than having PTSD when the spasm severity determined by the matching processor 22 is frequently high in winter.

The structure of the physical condition diagnoser 13 is not limited to the above example and may be any structure that can determine the physical condition index of the subject organism. In an example, the physical condition diagnoser 13 may include the matching processor 22 alone. In another example, the physical condition diagnoser 13 may include the matching processor 22 and the model diagnoser 24.

The output device 53 may output a control signal corresponding to the diagnosis results for an external device. In an example, when the subject organism is a livestock animal, the output device 53 outputs a control signal corresponding to the diagnosis results to an external device that improves the environment of the livestock barn, such as an air conditioner, a blower, or a feeder.

In another example, when the subject organism is a human who exercises using an exercise machine, the output device 53 outputs a control signal corresponding to the physical condition index included in the diagnosis results to the exercise machine. For example, when the diagnosis results indicate a lower health level as an example of the physical condition index, the output device 53 transmits a control signal for reducing the exercise load to the exercise machine.

In another example, when the subject organism is a human, the output device 53 outputs a control signal corresponding to the diagnosis results to an ordering device that places orders for, for example, medicines or supplements. When the diagnosis results indicate the possibility of a disease, for example, the physical condition diagnoser 13 transmits a control signal indicating an order of a medicine or a supplement for the disease to the ordering device.

Each of the physical condition diagnosis systems 100 and 101 may be implemented in any manner that can diagnose the physical condition of the subject organism. In an example, the physical condition diagnosis device 3 illustrated in FIG. 12 may be partially or entirely incorporated in the wearable device 56, together with the temperature sensors 51a and 51b, the activity sensors 52a and 52b, and the environment sensor 55. Each of the physical condition diagnosis devices 1, 2, 4, and 5 may also be partially or entirely incorporated in the wearable device 56.

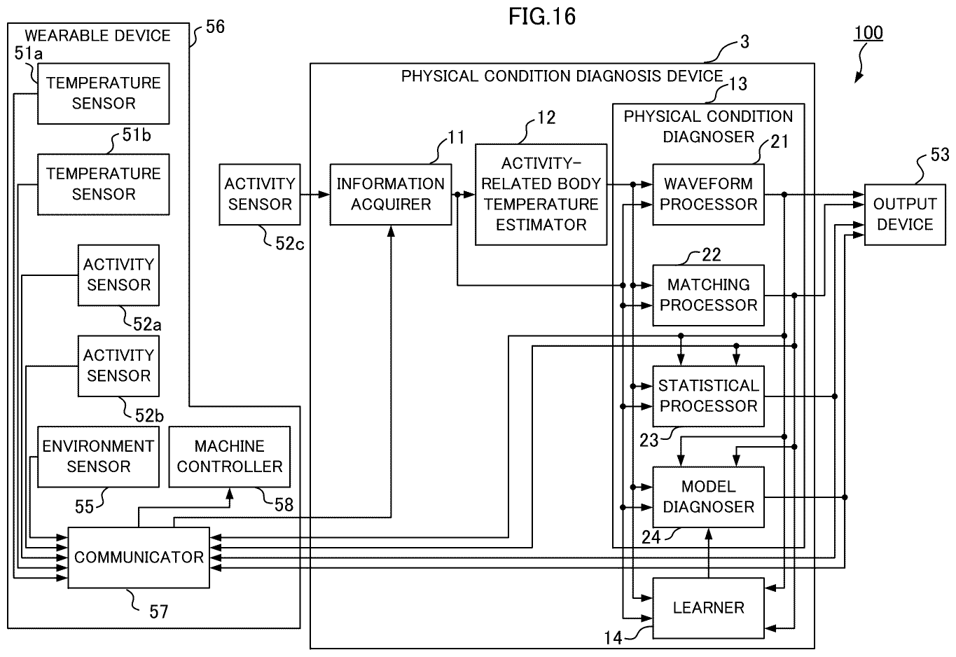

The wearable device 56 may transmit and receive information to and from the physical condition diagnosis device 3. More specifically, the wearable device 56 may acquire the physical condition index from the physical condition diagnoser 13. The communicator 57 included in the wearable device 56 illustrated in FIG. 16 acquires the physical condition index from the physical condition diagnoser 13 included in the physical condition diagnosis device 3. The wearable device 56 includes a machine controller 58 that controls the exercise machine used by the subject organism based on the physical condition index received by the communicator 57.

More specifically, the communicator 57 receives the physical condition index from at least one of the waveform processor 21, the matching processor 22, the statistical processor 23, or the model diagnoser 24 included in the physical condition diagnoser 13. The communicator 57 transmits the received physical condition index to the machine controller 58. For the subject organism exercising using an exercise machine, the machine controller 58 controls the exercise machine based on the exercise load corresponding to the physical condition index determined by the physical condition diagnoser 13 included in the physical condition diagnosis device 3 and received by the communicator 57. For example, when the health level as an example of the physical condition index received by the communicator 57 decreases, the machine controller 58 transmits a control signal for reducing the exercise load to the exercise machine.

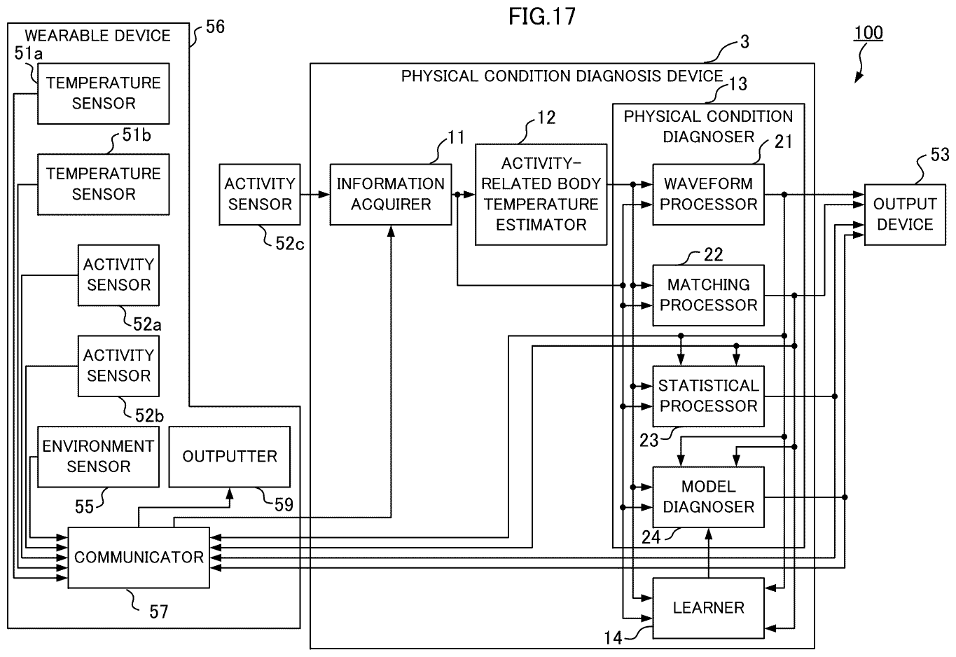

In another example, the wearable device 56 illustrated in FIG. 17 includes an outputter 59 that outputs information about the exercise load corresponding to the physical condition index received by the communicator 57 through, for example, screen display or sound. For example, the outputter 59 suggests a lower exercise load when the health level decreases, and suggests a higher exercise load when the health level increases. The exercise load is, for example, the running pace, the gradient, or the weight of an exercise tool.

In another example, the wearable device 56 illustrated in FIG. 16 or FIG. 17 may acquire the diagnosis results from the output device 53. In some embodiments, the output device 53 may be mounted on the wearable device 56.

The hardware configuration and the flowcharts described above are mere examples, and may be changed or modified as appropriate. The main portions including the processor 61, the memory 62, and the interface 63 to perform the control process can be implemented with a common computer system, rather than with a dedicated system. For example, a computer program executable to perform the above operation may be stored in a non-transitory computer-readable recording medium, such as a flexible disk, a compact disc ROM (CD-ROM), or a DVD-ROM for distribution. The computer program may be installed in a computer to provide each of the physical condition diagnosis devices 1 to 5 that performs the above processing. In some embodiments, the computer program may be stored in a storage device included in a server device on a communication network, and may be downloaded by a common computer system to implement each of the physical condition diagnosis devices 1 to 5.

When the functions of each of the physical condition diagnosis devices 1 to 5 are implemented partially by an operating system (OS) and partially by an application program, or through cooperation between the OS and the application program, portions of the application program alone may be stored in a non-transitory recording medium or a storage device.

The computer program may be superimposed on the carrier wave and distributed through the communication network. For example, the computer program may be posted on a bulletin board system (BBS) on the communication network to be provided through the communication network. The computer program may be activated and executed under the control of the OS in the same manner as another application program to perform the above processing.

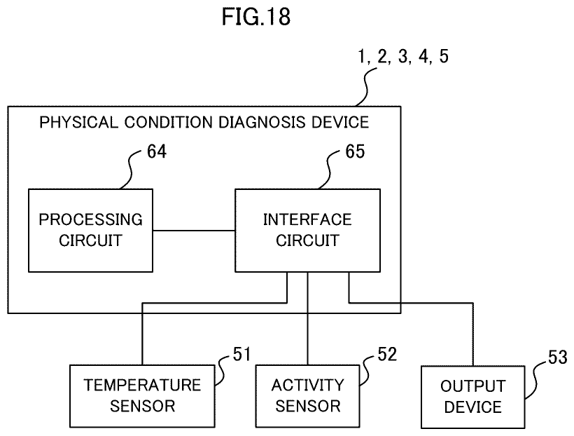

The hardware configurations of the physical condition diagnosis devices 1 to 5 are not limited to the examples described above. Each of the physical condition diagnosis devices 1 to 5 may be implemented by a processing circuit 64 as illustrated in FIG. 18. The processing circuit 64 is connected to, for example, the temperature sensor 51, the activity sensor 52, and the output device 53 through an interface circuit 65. When the processing circuit 64 is dedicated hardware, the processing circuit 64 is, for example, a single circuit, a complex circuit, a programmed processor, a parallel programmed processor, an application-specific integrated circuit (ASIC), a field-programmable gate array (FPGA), or a combination of two or more of these. The components of each of the physical condition diagnosis devices 1 to 5 may be implemented by individual processing circuits 64 or by a common processing circuit 64.

Each of the physical condition diagnosis devices 1 to 5 may have some functions implemented by dedicated hardware, and other functions implemented by software or firmware. For example, the information acquirer 11 may be implemented by the processing circuit 64 illustrated in FIG. 18, and the activity-related body temperature estimator 12 and the physical condition diagnoser 13 may be implemented by the processor 61 illustrated in FIG. 2 reading and executing the programs stored in the memory 62.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

### REFERENCE SIGNS LIST

1, 2, 3, 4, 5 Physical condition diagnosis device
11 Information acquirer
12 Activity-related body temperature estimator
13 Physical condition diagnoser
14 Learner
21 Waveform processor
22 Matching processor
23 Statistical processor
24 Model diagnoser
31 Model determination device
32 Physical condition model determiner
33 Temperature model determiner
51, 51a, 51b Temperature sensor
52, 52a, 52b, 52c Activity sensor
53 Output device
54 Screen
54a, 54b, 54c, 54d, 54e, 54f, 54g, 54h, 54i, 54j Display area
55 Environment sensor
56 Wearable device
57 Communicator
58 Machine controller
59 Outputter
60 Bus
61 Processor
62 Memory
63 Interface
64 Processing circuit
65 Interface circuit
81, 82 Part
91, 92 Heat source
100, 101 Physical condition diagnosis system

1. A physical condition diagnosis device, comprising:
information acquiring circuitry to acquire vital sign information and variation factor information, the vital sign information including temperature information of at least one part of a subject organism to undergo physical condition diagnosis, the variation factor information including activity information indicating an activity of the subject organism causing temperature variations in the at least one part;
activity-related body temperature estimating circuitry to determine, based on the vital sign information, the variation factor information, and a heat transfer path in a body of the subject organism, activity-related body temperature data indicating the temperature variations in an organ in the body of the subject organism caused by the activity, the heat transfer path being determined based on heat production and heat dissipation in the body of the subject organism; and
physical condition diagnosing circuitry to determine a physical condition index indicating a physical condition of the subject organism based on the activity-related body temperature data.

2. The physical condition diagnosis device according to claim 1, wherein
the information acquiring circuitry acquires the activity information including an activity index being a numerical value indicating the activity of the subject organism.

3. The physical condition diagnosis device according to claim 1, wherein
the information acquiring circuitry acquires the activity information including type information indicating a type of the activity of the subject organism.

4. The physical condition diagnosis device according to claim 1, wherein
the information acquiring circuitry acquires the variation factor information including the activity information and environmental information, and the environmental information indicates an environment surrounding the subject organism.

5. (canceled)

6. The physical condition diagnosis device according to claim 1, wherein
the activity-related body temperature estimating circuitry acquires a temperature model for deriving the activity-related body temperature data from the vital sign information and the variation factor information, and inputs the vital sign information and the variation factor information acquired by the information acquiring circuitry into the temperature model to determine the activity-related body temperature data indicating the temperature variations caused by the activity indicated by the activity information included in the variation factor information, and the temperature model is determined based on association between the vital sign information, the variation factor information, and the activity-related body temperature data.

7. The physical condition diagnosis device according to claim 1, wherein
the activity-related body temperature estimating circuitry determines, based on the vital sign information and the variation factor information, a plurality of pieces of the activity-related body temperature data different from each other in at least one of a target part or the activity.

8. (canceled)

9. The physical condition diagnosis device according to claim 1, wherein
the physical condition diagnosing circuitry determines the physical condition index indicating at least one of whether the subject organism is in a healthy condition, whether the subject organism has a disease, or a health level of the subject organism indicated by a numerical value.

10. The physical condition diagnosis device according to claim 1, wherein
the physical condition diagnosing circuitry determines the physical condition index based on at least one of the vital sign information or the variation factor information and based on the activity-related body temperature data.

11. The physical condition diagnosis device according to claim 1, wherein
the physical condition diagnosing circuitry includes waveform processing circuitry to determine the physical condition index based on at least one of a phase characteristic, an amplitude, a frequency spectrum, or a waveform of waveform data of the activity-related body temperature data.

12. The physical condition diagnosis device according to claim 1, wherein
the physical condition diagnosing circuitry includes matching processing circuitry to determine the physical condition index by pattern matching between a pattern defined based on the physical condition of the subject organism and the activity-related body temperature data.

13. (canceled)

14. The physical condition diagnosis device according to claim 1, wherein
the physical condition diagnosing circuitry outputs a result of physical condition diagnosis for the subject organism, and the result of physical condition diagnosis includes the physical condition index and at least one of the vital sign information, the variation factor information, or the activity-related body temperature data.

15. The physical condition diagnosis device according to claim 1, wherein
the physical condition diagnosing circuitry further includes model diagnosing circuitry to acquire a physical condition model for deriving the physical condition index from the activity-related body temperature data and the variation factor information and to input the activity-related body temperature data determined by the activity-related body temperature estimating circuitry and the variation factor information acquired by the information acquiring circuitry into the physical condition model to determine the physical condition index of the subject organism, and the physical condition model is determined based on association between the activity-related body temperature data, the variation factor information, and the physical condition index.

16. The physical condition diagnosis device according to claim 1, further comprising:
learning circuitry to determine a physical condition model for deriving the physical condition index from the activity-related body temperature data and the variation factor information, and the physical condition model is determined based on association between the activity-related body temperature data, the variation factor information, and the physical condition index.

17. A wearable device wearable by a subject organism to undergo physical condition diagnosis, the wearable device being a device for transmitting and receiving information to and from the physical condition diagnosis device according to claim 1, the wearable device comprising:
a temperature sensor to measure a temperature of at least one part of the subject organism;
an activity sensor to generate an activity index being a numerical value indicating an activity of the subject organism causing temperature variations in the at least one part;
communicating circuitry to transmit a measurement value acquired by the temperature sensor and the activity index generated by the activity sensor to the physical condition diagnosis device, and receive, from the physical condition diagnosis device, the physical condition index determined by the physical condition diagnosing circuitry included in the physical condition diagnosis device; and
machine controlling circuitry to control, for the subject organism exercising using an exercise machine, the exercise machine based on an exercise load corresponding to the physical condition index determined by the physical condition diagnosing circuitry included in the physical condition diagnosis device and received by the communicating circuitry.

18. A wearable device wearable by a subject organism to undergo physical condition diagnosis, the wearable device being a device for transmitting and receiving information to and from the physical condition diagnosis device according to claim 1, the wearable device comprising:
a temperature sensor to measure a temperature of at least one part of the subject organism;
an activity sensor to generate an activity index being a numerical value indicating an activity of the subject organism causing temperature variations in the at least one part;
communicating circuitry to transmit a measurement value acquired by the temperature sensor and the activity index generated by the activity sensor to the physical condition diagnosis device, and receive, from the physical condition diagnosis device, the physical condition index determined by the physical condition diagnosing circuitry included in the physical condition diagnosis device; and
outputting circuitry to output, for the subject organism exercising, information about an exercise load corresponding to the physical condition index determined by the physical condition diagnosing circuitry included in the physical condition diagnosis device and received by the communicating circuitry.

19. A physical condition diagnosis system, comprising:
a temperature sensor to measure a temperature of at least one part of a subject organism to undergo physical condition diagnosis; and
the physical condition diagnosis device according to claim 1,
wherein the information acquiring circuitry included in the physical condition diagnosis device acquires a measurement result from the temperature sensor as the temperature information.

20. The physical condition diagnosis system according to claim 19, further comprising:
an activity sensor to generate an activity index being a numerical value indicating an activity of the subject organism causing temperature variations in the at least one part,
wherein the information acquiring circuitry included in the physical condition diagnosis device acquires the activity information including type information and the activity index, the type information indicates a type of the activity associated with the activity sensor, and the activity index is generated by the activity sensor.

21.-23. (canceled)

24. A model determination device, comprising:
temperature model determining circuitry to learn association between vital sign information, variation factor information, and activity-related body temperature data, and determine a temperature model for deriving the activity-related body temperature data from the vital sign information and the variation factor information, the vital sign information including temperature information of at least one part of a subject organism to undergo physical condition diagnosis, the variation factor information including activity information indicating an activity of the subject organism causing temperature variations in an organ of the subject organism, the activity-related body temperature data indicating the temperature variations in the at least one part caused by the activity.

25. The model determination device according to claim 24, further comprising:
physical condition model determining circuitry to learn association between the activity-related body temperature data, the variation factor information, and a physical condition index indicating a physical condition of the subject organism, and determine a physical condition model for deriving the physical condition index from the activity-related body temperature data and the variation factor information.

26. A physical condition diagnosis method, comprising:
acquiring vital sign information and variation factor information, the vital sign information including temperature information of at least one part of a subject organism to undergo physical condition diagnosis, the variation factor information including activity information indicating an activity of the subject organism causing temperature variations in the at least one part;
determining, based on the vital sign information, the variation factor information, and a heat transfer path in a body of the subject organism, activity-related body temperature data indicating the temperature variations in an organ in the body of the subject organism caused by the activity, the heat transfer path being determined based on heat production and heat dissipation in the body of the subject organism; and
determining a physical condition index indicating a physical condition of the subject organism based on the activity-related body temperature data.

27. (canceled)
